(19) United States
(12) Patent Application Publication
(10) Pub. No.: US 2025/0302652 A1
(43) Pub. Date: Oct. 2, 2025

(54) Versatile Knee Brace

(71) Applicant: Daniel Nissan Weizel, Tel Aviv (IL)

(72) Inventor: Daniel Nissan Weizel, Tel Aviv (IL)

(21) Appl. No.: 18/616,379

(22) Filed: Mar. 26, 2024

(51) Int. Cl. A61F 5/01

(52) U.S. Cl. CPC A61F 5/0125

(57) ABSTRACT

The invention is a knee-brace with stabilizer-containment sleeves on each side that allow; stabilizers with varying degrees of stabilization to be swapped in and out to accommodate the stabilization required at different stages of healing.

### TECHNICAL FIELD

The invention is knee brace operative to support a wearer's knee.

### BACKGROUND OF INVENTION

The human knee joint comprises several bones, ligaments and tendons. It is an astounding combination that provides a variety of freedoms of motion while supporting a person's body weight of all body mass above it, while standing, walking or running.

Knee joints, because of the complexity of the structures and attachments, can be injured by excess lateral (sideways) motion. With age, the knee-joint components may wear and become weakened making injury even more likely.

Knee braces are devices that surround the area of the knee providing support and optionally limiting motion in lateral directions. When a knee has become weakened, injured or has been operated upon, many people opt to use knee braces for added protection against further injury, or while healing from injury or surgery.

Knee braces are available in a variety of forms. For example, after surgery or injury, a knee brace with significant restriction of lateral motion may be worn. As healing takes place, a different knee brace may be selected that offers more freedom of movement, lower weight, and greater comfort. A user may ultimately opt for an elastic, stretchable device that stays in place around the knee area but provides no additional stabilization against lateral motion, such as a more rigid stabilizer might provide.

In most cases, after severe injury or knee surgery, users go through a progression of different knee braces before opting to dispense with brace support. As a result, users often end up purchasing a succession of braces because each one is limited to a specific support outcome.

### BRIEF DESCRIPTION OF INVENTION

The invention herein disclosed is a knee brace the combines the light-weight of an elastic, stretchable knee brace with the support of lateral-motion stabilization. Moreover, because of its structure, it may replace a succession of braces with a single brace that is versatile in terms of degrees of stabilization.

The versatility is a direct result of stabilizer sleeves structures that are strategically positioned for optimal lateral support and may be easily and quickly adjusted for varying degrees of support using a variety of stabilizer element structures that can be inserted or removed from these sleeves.

A user may start out with the brace using a high degree of stabilization right after injury or surgery, then remove and replace it with a succession of lighter-duty stabilization elements. Ultimately, the brace may be worn with no added stabilizers, and even the stabilizer sleeves may be removed.

### BRIEF DESCRIPTION OF DRAWINGS

FIG. 1 shows a human knee comprised of various bones, tendons and ligaments.

FIG. 2 shows the types of motion the knee joint is capable of supporting.

FIG. 3 shows an exemplary knee brace with its built-in stabilizers.

FIG. 4 shows an exemplary knee brace comprising an elastic, stretchable knee-surrounding sleeve with no additional stabilization.

FIG. 5 shows a perspective view of an embodiment of the invention showing its knee-surrounding sleeve and stabilizer sleeves structures.

FIG. 6 shows a front view of an embodiment of the invention showing the stabilizer sleeves located on both the right and left sides of the knee-surrounding sleeve.

FIG. 7 shows the user's left-side view of the brace embodiment showing the position of the left-side stabilizer sleeve for optimal support of the knee's leftward lateral motion.

FIG. 8 shows the user's right-side view of the brace embodiment showing the position of the right-side stabilizer sleeve for optimal support of the knee's rightward lateral motion.

FIG. 9 depicts one of several different strength stabilizers being inserted into one of the knee brace's stabilizer sleeves.

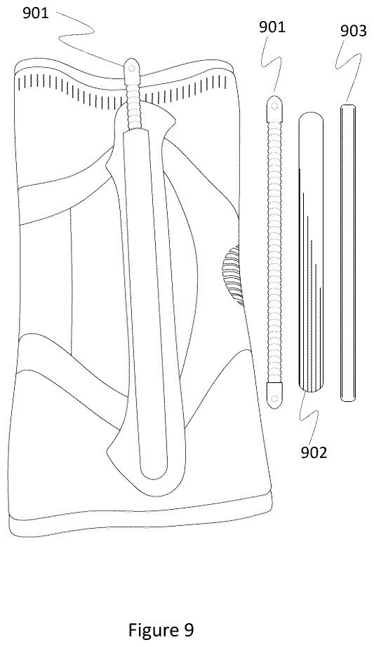

FIG. 10 depicts a removable stabilizer sleeve using a hook-and-loop like mounting fixture.

### DETAILED DESCRIPTION OF INVENTION

A human knee is a marvelous joint comprising a set of bones, ligaments and tendons that give it significant freedom of motion whether standing, walking or running. In addition, it is robust enough to do all this while supporting the weight of all body mass above the knee while standing, walking or running.

The knee is a complex mechanical device made up of rigid structures (bones) and flexible attaching fixtures (ligaments and tendons). Like any mechanical device, if stressed beyond its limits, it may be damaged. Depending on the nature of the damaging injury, supporting braces may be prescribed, or, perhaps surgery must be done followed by use of supporting braces.

A type of brace that permits walking motion but limits lateral motion is known as a functional brace. Functional braces may be made of a combination of metallic structures and compressible structures that enable it to be mounted in place surrounding the knee, securely held in place, and providing stabilizing structures that limit motion to the sides.

Right after an injury or surgery, most people need significant support against lateral motion. As healing and rehabilitation takes place, the degree of stabilization may be reduced to provide increased freedom of motion, lower weight, and greater comfort.

For many people with knee injuries or post-surgery convalescence, it may be necessary to acquire more than one brace to facilitate the gradual reduction in support and increase in lateral motion and comfort.

Usually, the last type of brace that a person may use is made essentially of elastic, stretchable material that slips over the foot, is pulled up over the calf, and surrounds the knee with a modest degree of support, low weight and reduced discomfort. The initial brace may be a heavy, essentially metallic, device that provides significant support but may be heavy and uncomfortable.

The invention herein disclosed is a versatile knee brace operative to provide the low weight and structure of the elastic, stretchable type of brace with the stabilizing elements of a heavier-duty brace. Moreover, rather than using a fixed stabilizing element, the device uses stabilizer sleeves allowing insertion and removable of elements of various support characteristics. As such, the same brace can be used for an extended period of time while providing the various support needs at different stages of healing.

The following descriptions and drawings a meant to further describe the context and specifics of the invention. These drawings are meant to be exemplary and should not be read as limiting the scope of claims.

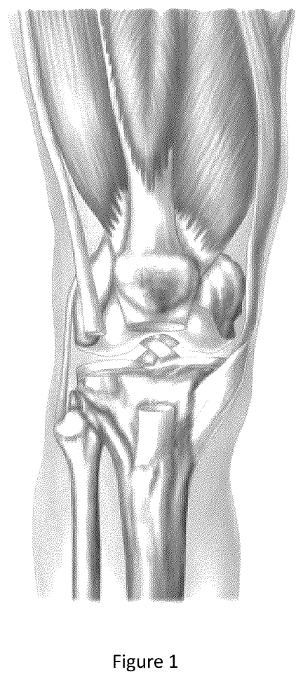

In FIG. 1, in interior view of a human knee is shown comprising multiple bones, ligaments and tendons. The bones provide rigid structures and the ligaments and tendons provide flexible attachment structures.

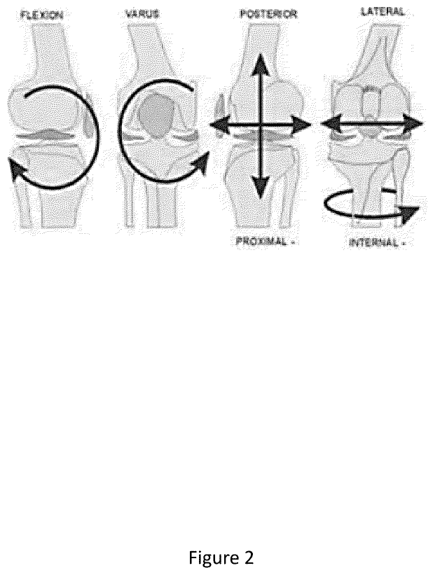

As shown in FIG. 2, the knee structure serves to allow a variety of motions. Usually knee injuries occur when a knee is pushed beyond its limits in lateral directions. For example, in sporting games where one player is hit by another, from the side, forcing the knee to bend laterally, torn ligaments and tendons may result.

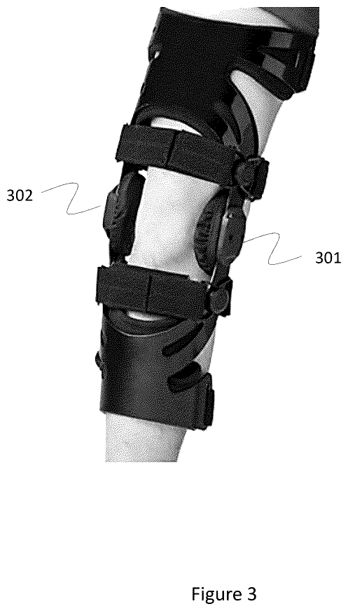

FIG. 3 shows an exemplary knee brace that is made of a combination of rigid (metallic) elements, compressible elements (rubber or foam) and adjustable strap elements. It is meant to be positioned above and below the knee, held in place by its rigid structures, and straps, and limit lateral motion through its side stabilizers (301 and 302). Note that the stabilizers are essentially mirror images of one another. Stabilizer 301 is designed to be on the user's left side, and 302 on the right side.

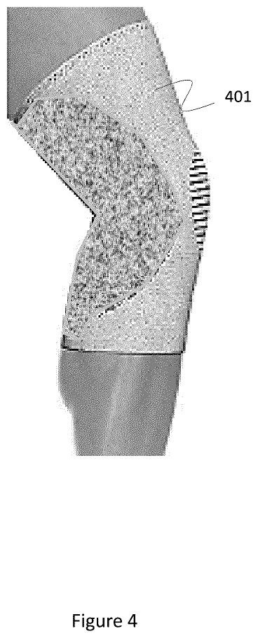

FIG. 4 shows a knee brace comprising elastic, stretchable fabric forming a knee-surround sleeve (401). Such braces are designed to be pulled over a foot and calf, into position surrounding the knee, and held in place by the compression of the elastic once positioning is completed. The sleeve material will provide some support but the focus is on low weight and greater comfort.

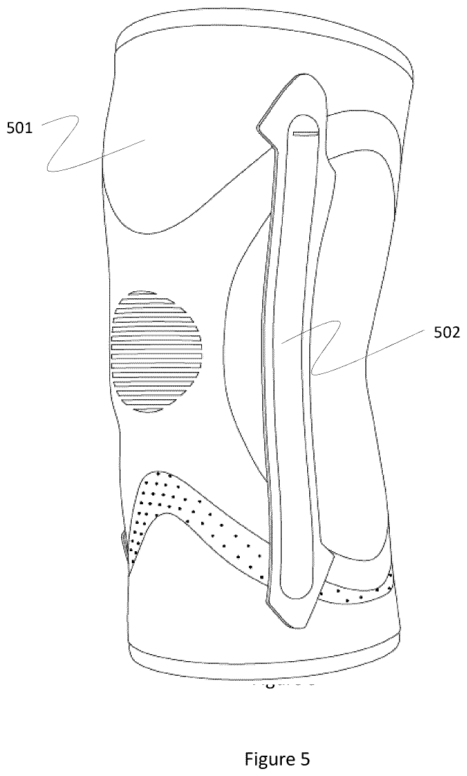

In one embodiment of the invention, FIG. 5 shows a knee-surrounding sleeve (501) and a left-side stabilizer-containment sleeve (502). The stabilizer-containment sleeve is operative to conform in shape to the knee-surrounding sleeve's shape at the position of attachment, and is made of material that is somewhat less flexible than the fabric of the knee-surrounding sleeve. That said, the stabilizer sleeve is not rigid and will bend with lateral knee motion. Its primary purpose is to provide a fixture for inserting a stabilizer and holding it in place at an optimal support position.

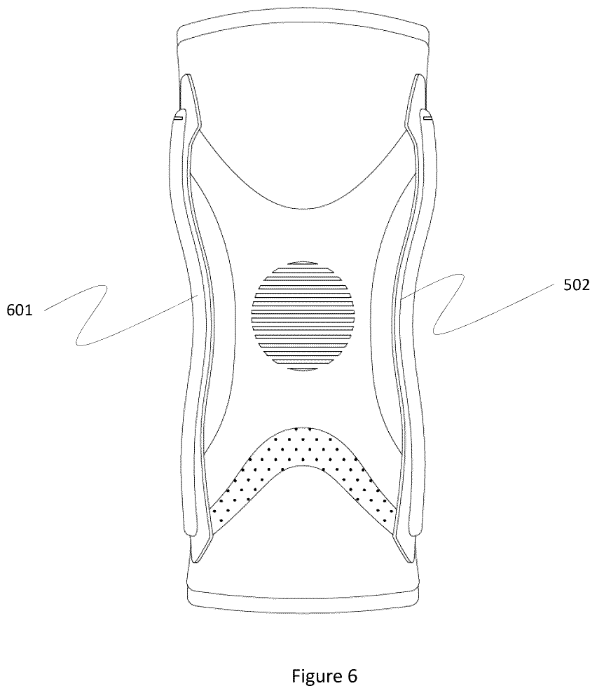

FIG. 6 shows a front view of the knee-surrounding sleeve with the user's left-side (502) and right-side (601) stabilizer-containment sleeves on their respective sides. Note that 502 and 601 are mirror-image versions of each other.

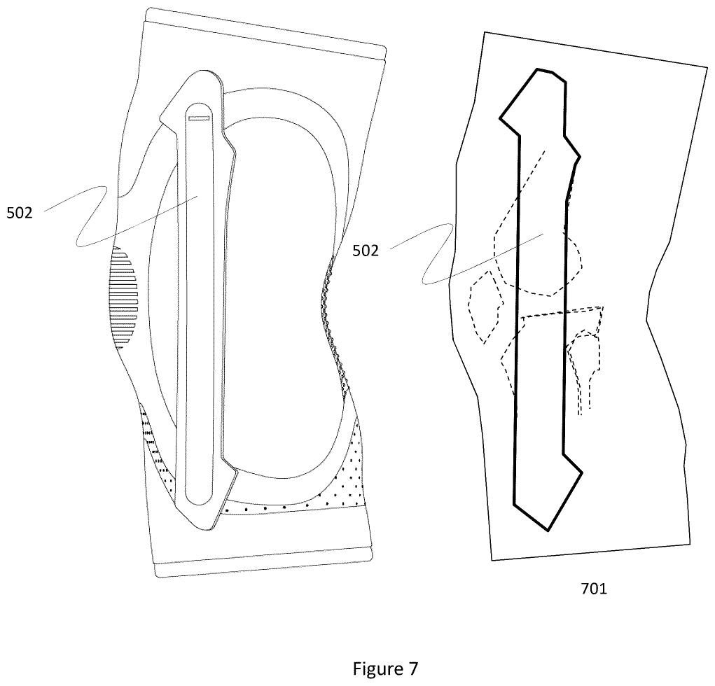

In FIG. 7, the left-side stabilizer-containment sleeve (502) is positioned closer to the front of the knee-surrounding sleeve on its left side. This corresponds to the optimal position relative to the knee bone positions (701), providing optimum lateral-motion stabilization on the left side.

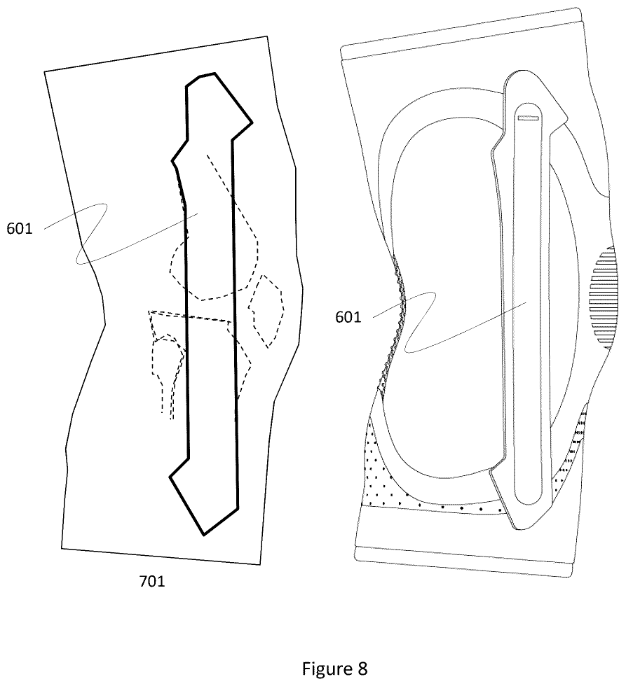

In FIG. 8, the right-side stabilizer-containment sleeve is positioned closer to the front of the knee-surrounding sleeve on its right side. This corresponds to the optimal position relative to the knee-bone positions (701), providing optimum lateral-motion stabilization on the right side.

The stabilizer sleeves (502 and 601) are operative to allow a variety of stabilizers to be inserted through the upper portion (901) as shown. This is exemplary. The sleeve can also be operative to allow insertion from the bottom, the front, or the sides. Front and side openings would be longer than top and bottom openings and would include a means of closure such as, but not limited to, snaps, buttons or zippers. An exemplary variety of stabilizers are shown (901, 902 and 903). This are shown with different profiles but should not be seen as limiting the size and shape of such stabilizers. The length and width would be limited by the stabilizer sleeve's tubular enclosure's length and width, but the stabilizers can be made in a variety of shapes. Stabilizer material could be any material that provides the intended stabilization characteristics. A stabilizer could be rigid to provide a high degree of lateral-motion restriction, or be somewhat flexible to provide a lesser degree of lateral-motion restriction. The sleeve plus the variety of stabilizers enables this knee-brace invention to provide variable levels of stabilization that could suffice from early injury/surgery time to minimal stabilization at later stages of healing. As a result, a single knee brace could provide all stabilization that is required at any stage of knee injury/surgery recovery.

The stabilizer-containment sleeves in these exemplary cases are shown as separate components of the knee-brace invention. They could also be integral components, integrated into the structure of the knee-surrounding sleeve, and operative to allow insertion and removal of a variety of stabilizer elements.

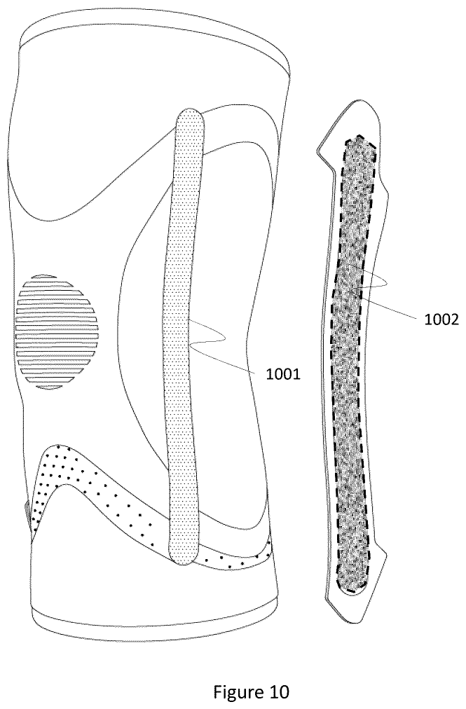

The stabilizer-containment sleeves could be permanently attached in position on the knee-surrounding sleeve making the stabilizer-containment sleeves a non-removable component. As shown in FIG. 10, the stabilizer sleeves could be attached to the knee-surrounding sleeve using a means of attachment, such as hook-and-loop tape fixtures (1001 and 1002) that are affixed to the surface of the knee-surrounding sleeve (1001) and the back portion of the stabilizer sleeve (1002). Other means of attachment may be used that provide sufficient adhesion when attached to prevent detachment during normal use of the brace.

A method of use for the knee-brace invention would involve first pulling the knee-surrounding sleeve over foot and calf to a position wherein the top of the sleeve is above the knee, and the bottom of the sleeve is below the knee. Next, the sleeve would be rotated such that each stabilizer-containment sleeve was positioned approximately the same distance back from the front of the user's knee. Once properly positioned, depending upon the stage of healing and stabilization required, a pair of stabilizers meeting the required stabilization characteristics is then inserted into the tubular enclosure of each stabilizer-containment sleeve. As reduced stabilization is needed during later stages of healing, a high-stabilization pair of stabilizers may be removed and a lighter-stabilization pair is then inserted. As such, the single knee brace may accommodate the varying stabilization needs during most or all stages of injury or surgery recovery.

All drawings and descriptions are meant to be exemplary and should not be read as limiting the scope of the invention. The knee-surrounding sleeve may be implemented using any fabric with sufficient elasticity and stretch. The stabilizer sleeves may be made of any material that can be formed into the intended shape and size operative to allow insertion and removal of compatible stabilizer elements, which when inserted will be held essentially in place with minimal horizontal or vertical displacement. The stabilizer sleeve material should be more inflexible than the knee-surrounding sleeve material but should not be rigid thereby allowing a non-rigid stabilizer to flex, as needed, to be provide intended levels of lateral-motion support.

What is claimed is:

1. A knee-brace comprising:
a knee-surrounding sleeve;
a right-side stabilizer-containment sleeve;
a left-side stabilizer-containment sleeve; and
stabilizer elements operative to be inserted into the right-side and left-side stabilizer-containment sleeves.

2. A claim as in claim 1 wherein:
the knee-surrounding sleeve is made of elastic, stretchable fabric.

3. A claim as in claim 1 wherein:
the right-side and left-side stabilizer-containment sleeves are operative to enclose one of the stabilizer elements inside a tubular enclosure.

4. A claim as in claim 3 wherein:
the right-side and left-side stabilizer-containment sleeves, when affixed to the knee-surrounding sleeve, are oriented such that their tubular containment axes are essentially vertical when a person is wearing the brace and standing.

5. A claim as in claim 1 wherein:
the stabilizer elements are operative to be inserted into the left-side and right-side stabilizer-containment sleeves' tubular enclosures and the stabilizers' lengths and girths will fit completely inside these tubular enclosures.

6. A claim as in claim 1 wherein:
the stabilizer elements are rigid and do not flex.

7. A claim as in claim 1 wherein:
the stabilizer elements are semi-rigid and capable of flexing.

8. A claim as in claim 1 wherein:
the right-side and left-side stabilizer-containment sleeves are permanently affixed to the knee-surrounding sleeve.

9. A claim as in claim 1 wherein:
the right-side and left-side stabilizer-containment sleeves are attached to, and detached from, the knee-surrounding sleeve using an adhesion fixture.

10. A claim as in claim 1 wherein:
the right-side and left-side stabilizer-containment sleeves are positioned on the knee-surrounding sleeve such that they are on opposite sides of the knee-surrounding sleeve directly adjacent to the position of a surrounded knee's bones interface.

11. A claim as in claim 5 wherein:
the stabilizer elements are inserted through an opening in the top of the stabilizer-containment sleeves, the top being the uppermost portion of the attached stabilizer-containment sleeves when a user is standing.

12. A claim as in claim 5 wherein:
the stabilizer elements are inserted through an opening in the bottom of the stabilizer-containment sleeves, the bottom being the lowest portion of the attached stabilizer-containment sleeves when a user is standing.

13. A claim as in claim 5 wherein:
the stabilizer elements are inserted through an opening in the side of the stabilizer-containment sleeves, the side opening being parallel to the axis of the tubular enclosure.
